(19) United States
(12) Patent Application Publication
(10) Pub. No.: US 2012/0136028 A1
(43) Pub. Date: May 31, 2012

(54) Methods Of Diminishing Co-Abuse Potential

(75) Inventors: Vikram Khetani, Short Hills, NJ (US); Herbert Faleck, West Orange, NJ (US)

(73) Assignee: CELGENE CORPORATION, Summit, NJ (US)

(21) Appl. No.: 13/370,663

(22) Filed: Feb. 10, 2012

Related U.S. Application Data
(63) Continuation of application No. 12/269,471, filed on Nov. 12, 2008.
Application No. 12/269,471 is a continuation of application No. 10/832,210, filed on Apr. 26, 2004, now abandoned.

(51) Int. Cl. A61K 31/4458; A61P 25/00

(52) U.S. Cl. 514/317

(57) ABSTRACT

Methods of diminishing or eliminating the co-abuse of a methylphenidate drug comprising identifying a patient or patient group suspected or likely to abuse said methylphenidate drug in combination with a substance known or suspected to give rise to l-ethylphenidate or psychotropic effect when ingested in the combination and making available to said patent or patient group said methylphenidate drug substantially free of l-threo methylphenidate.

### CROSS-REFERENCE TO RELATED APPLICATION

This application is a continuation of U.S. application Ser. No. 12/269,471, filed Nov. 12, 2008, which is a continuation of U.S. application Ser. No. 10/832,210, filed Apr. 26, 2004, now abandoned, the entireties of which are incorporated herein.

### FIELD OF THE INVENTION

This invention relates to methods of reducing or eliminating the co-abuse of methylphenidate drugs with abusable substances such as alcohol, cocaine, opioids or nicotine. Also disclosed are methods to reduce adverse effects associated with co-administration of phenidate drugs and substances such as alcohol or other central nervous system active agents in subjects undergoing treatment for ADHD and other disorders where phenidate products are administered.

### BACKGROUND OF THE INVENTION

Methylphenidate has been known to be co-administered with alcohol and other substances by substance abusers. In a recent survey, three percent of college students abuse methylphenidate. According to Teter, et al., Pharmacotherapy, 2003; 23:609-17, ninety-eight percent of these illicit methylphenidate users are also binge drinkers. Barrett, et al., J. Clin Psychopharmacol, 2002; 22:633-4, discloses that the oral mode of administration of methylphenidate is becoming more common than intravenous or intranasal modes when used concomitantly with alcohol. There are some theories for the co-abuse of methylphenidate and alcohol. They include alteration and enhancement in psychotropic effects, production of desirable effects characterized by increased euphoria and energy as well as diminished sense of drunkenness, and an experience likened to “low-grade cocaine” or “diet coke.” Another theory is that children who grow up in the habit of taking stimulants for their ADHD are more likely to take illegal stimulants such as cocaine or methamphetamine. There is a need for a treatment that will not yield the addictive euphoric effects experienced when methylphenidate is co-abused with other stimulants.

There is also a need for a method of reducing the adverse effects associated with co-administration of phenidate drug and substances such as alcohol, or other central nervous system active agents in patients undergoing treatment for ADHD. According to Sabri, et al., Alcohol and Alcoholism, 2003; 38:352-6, alcohol dependence in subjects with childhood ADHD starts early and is resistant to treatment. Co-morbid substance abuse is more prevalent in ADHD subjects. Adults with ADHD are known to have co-morbid alcohol problems and an elevated risk of substance use disorders.

Subjects using phenidate drugs to treat cancer-related fatigue and cognitive disorders also exhibit the potential to co-abuse phenidate. These subjects are more likely to take medications such as opioid analgesics for pain. These subjects are also more likely to co-administer methylphenidate and alcohol. A safer drug is needed for these subjects compared to other formulations containing methylphenidate, particularly the racemic mixture.

It has also been found that subjects using phenidate drugs as adjuvants to substances used to treat pain conditions including but not limited to Complex Regional Pain Syndrome, low back pain, fibromyalgia, radiculopathy, peripheral neuropathy e.g. diabetic neuropathy, post-herpetic neuralgia, trigeminal neuralgia are more likely to be taking medications such as opioid analgesics for pain. These subjects are also more likely to co-administer methylphenidate and alcohol. Patients being treated for neurologic or neuropsychiatric disorders including but not limited to stroke, head trauma, and depression are also more likely to co-administer methylphenidate and alcohol.

Markowitz, et al., J. Clin. Psychopharmacol., 1999; 19:362-6, found that ethylphenidate was detected as a new metabolite in plasma after methylphenidate overdose with alcohol coingestion. Many methylphenidate products such as Concerta®, Ritalin®, and Metadate® carry a great co-abuse potential because ethylphenidate is a metabolite of these products. Markowitz, et al., Drug Metabolism and Disposition, 2000; 28:620-5 states that ethylphenidate was also detected in plasma and urine in human subjects after co-administration of a single dose of methylphenidate and ethanol. Ethylphenidate is known to have dopaminergic activity. It was found recently by Thomson, et al., 31st Annual Am. College of Clin. Pharmacol. Meeting Abstracts, 2002, that after co-administration of racemic methylphenidate and ethanol, l-methylphenidate transesterifies enantioselectively to l-ethylphenidate. Ethylphenidate metabolite was excreted in the urine primarily as the l-isomer. Methylphenidate was excreted primarily as the d-isomer. Ethylphenidate potentially contributes to the euphoric effects in co-abusers and adverse effects in patients with ADHD, cancer subjects and subjects with neurological or neuropsychiatric disorders.

An object of the present invention is to provide a means of treating patients who have an increased potential to co-abuse methylphenidate with illicit drugs such as cocaine and methamphetamine. It is also the object of the present invention to reduce potential adverse effects associated with co administration of phenidate drug and alcohol, opioids or other central nervous system active agents.

Another object of the present invention is to reduce adverse effects associated with co-administration of phenidate drugs and alcohol, opioids, nicotine, or other central nervous system active agents in subjects using phenidate drugs to treat cancer-related fatigue and cognitive disorders. The threo racemate (pair of enantiomers) of methylphenidate is a mild central nervous system stimulant with pharmacological activity qualitatively similar to that of amphetamines. It has been postulated that there are undesirable side effects associated with the use of the dl-threo racemate of methylphenidate including anorexia, weight loss, insomnia, dizziness and dysphoria. Also, the racemate is a Schedule II controlled substance that produces a euphoric effect when administered intravenously or through inhalation or ingestion, and thus carries a high potential for abuse. Recently, it has been discovered that the racemate may also lead to co-abuse of methylphenidate with other stimulants. Therefore, there is a need for treatments that administer compositions that will not interact with alcohol like racemic methylphenidate and will not produce adverse effects.

### SUMMARY OF THE INVENTION

The present invention provides methods of reducing co-abuse of a methylphenidate drug with a substance suspected to give rise to l-ethylphenidate when ingested in combination with the methylphenidate drug comprising identifying a patient or patient group suspected of abusing or likely to abuse the combination and providing to the patient or patient group a methylphenidate drug substantially free of l-threo methylphenidate. The methylphenidate drug may comprise dexmethylphenidate. There are also methods of reducing co-abuse of the combination of a methylphenidate drug with an addictive substance comprising identifying a patient or patient group suspected of abusing or likely to abuse the combination, and making available to the patient or patient group a methylphenidate drug substantially free of l-threo methylphenidate.

There are embodiments wherein the substance that gives rise to l-ethylphenidate may be ethyl alcohol. There are also embodiments where the addictive substance may be cocaine or cocaine derivative, an opioid, ethyl alcohol, a CNS active agent, or nicotine. It will be appreciated that the addictive substance may be known to produce a psychotropic effect. The drug may be made available to the patient or subject orally, intravenously, parenterally, via an aerosol or gaseous suspension, or transdermally. These dosage forms may comprise up to about 10 mg, 5 mg, or 3 mg of a methylphenidate drug that is substantially free of l-threo methylphenidate. The methods may comprise from about 0.1 mg to about 100 mg of said methylphenidate drug substantially free of l-threo methylphenidate. The patients are sometimes suspected of having ADD or ADHD.

There are also methods of reducing co-abuse of a methylphenidate drug with a substance suspected to give rise to l-ethylphenidate when ingested in combination with the methylphenidate drug comprising identifying a patient or patient group suspected of abusing or likely to abuse the combination and decreasing the amount of l-ethylphenidate produced by ingestion of the combination by making available said patient or patient group a methylphenidate drug substantially free of l-threo methylphenidate.

### DETAILED DESCRIPTION OF PREFERRED EMBODIMENTS

In accordance with the present invention, methods are provided to diminish the co-abuse of methylphenidate drugs with an addictive or abusable substance. One embodiment that may be preferred comprises identifying a patient or patient group suspected or likely to abuse a methylphenidate drug in combination with a substance known or suspected to give rise to l-ethylphenidate when ingested in the combination and making available to said patient or patient group said methylphenidate drug substantially free of l-threo methylphenidate.

Some embodiments of the present invention relate to a methylphenidate drug composition that reduces or eliminates the potential for co-abuse or the co-abuse itself. Co-administration of the methylphenidate drug and other abusable substances, such as but not restricted to alcohol, cocaine, or nicotine, does not produce the same psychotropic effects as racemic methylphenidate or amphetamines. As such, drug formulations containing compositions disclosed herein will unlikely be co-abused by substance abusers. Methylphenidate exists as four separate optical isomers as follows:

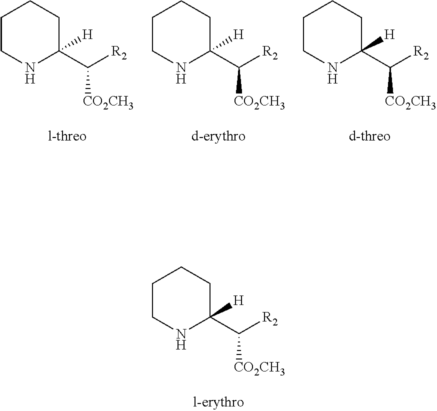

wherein R₂ is phenyl. Pharmaceutically acceptable salts are generally administered clinically. Other phenidate drugs, which also can be administered according to the invention, include those in which the methyl group in the above structures is replaced by C₂-C₄ alkyl and R₂ is optionally substituted with C₁-C₄ alkyl.

Clinically, the threo pair of enantiomers of methylphenidate hydrochloride is generally administered for the treatment of ADD and ADHD. The hydrochloride salt is commonly referred to simply as “methylphenidate.” Unless indicated otherwise, the term “methylphenidate” is used broadly herein to include methylphenidate and pharmaceutically acceptable salts thereof, including methylphenidate hydrochloride.

Although dl-threo-methylphenidate is generally used therapeutically, this racemate includes the l isomer which apparently makes no significant contribution to the pharmacological effectiveness of the drug, but likely contributes to the associated side effects. It is thus desirable to administer a dosage from substantially free of the l isomer. It has now been found that similar dosage forms may be used to diminish the co-abuse potential of methylphenidate.

In a further aspect, the present invention is directed to reducing the CNS adverse effects of phenidate drugs, exacerbated by substances such as, but not restricted to alcohol, opioid analgesics, nicotine, and other central nervous system active agents. More specifically, drug formulation containing dexmethylphenidate, also known as methylphenidate that is substantially free of the l-isomer, is a preferred mode of treating ADHD subjects, who have higher co-morbid alcohol dependence and higher risk of substance use disorders than the general population. These subjects are more likely to consume alcohol while on medication for ADHD. Furthermore, dexmethylphenidate is a preferred mode for treating patients with cancer-related fatigue and cognitive disorders or other neurological or neuropsychiatric disorders who are more likely to consume alcohol and co-administer other medications such as opioid analgesics for pain relief or other central nervous system active agents.

The drug formulations of some embodiments of the present invention are substantially free of l-threo-methylphenidate and erythro-methylphenidate. The reduction of co-abuse potential and adverse effects may be attributed to the absence of l-threo-isomer in some embodiments. Embodiments of the present invention have diminished co-abuse potential and reduced adverse effects when co-administered with alcohol. It will be appreciated that there may be several theories that explain this result. One may be that the dexmethylphenidate formulation is substantially free of l-threo-methylphenidate and erythro-methylphenidate.

The drug formulations of the present invention do not produce the euphoric effects when co-abused with other addictive substances. One feature of the present invention is that the drug does not produce adverse effects in ADHD or cancer patients who are also consuming alcohol or taking medications such as opioid analgesics.

Co-administration of racemic methylphenidate and alcohol has been known to produce psychotropic effects in humans. These adverse effects are potentially due to the production of l-ethylphenidate metabolite. One advantage of the present invention may be that co-administration of dexmethylphenidate and alcohol will not produce these effects. One difference between some embodiments of the present invention and similar methods of co-abuse reduction with racemic methylphenidate and alcohol is that dexmethylphenidate is substantially free of the l-isomer. Therefore, the l-ethylphenidate metabolite is not formed. L-ethylphenidate is potentially responsible for the euphoric and adverse effects.

The methods of the present invention may have a number of embodiments particular to the specific needs of one skilled in the art. In some methods, the drug provided to or administered to the patient comprises pharmaceutical unit dosage form that may have up to about 10 mg of dexmethylphenidate substantially free of l-threo methylphenidate. In other embodiments, the drug comprises up to about 5 mg of dexmethylphenidate or up to about 3 mg of said dexmethylphenidate both substantially free of l-threo methylphenidate. In some other embodiments the unit dosage form may be varied from about 0.1 mg to about 100 mg or greater of dexmethylphenidate, and yet accomplish a diminishing co-abuse effect.

The patient being treated may be made available or administered the dosage forms of the present invention by oral, intravenous, parenteral, aerosol or gaseous suspension, or transdermal means. Oral administration may be through capsule or tablets.

### Example

Subjects: Eight male Sprague-Dawley rats are maintained on a restricted feeding regimen to hold their weights in the range of 375-425 grams.

Apparatus: Experimental sessions are conducted in commercial, two-lever, rat operant chambers contained within sound- and light-attenuating cubicles. The chambers are equipped with pellet dispensers for 45-mg pellets. A stimulus light signals when the session is in progress.

Training: Subjects are used that had already been trained to discriminate coadministered ethanol/cocaine from saline. The rats have been trained to lever press for food reinforcement during daily 30-minute sessions. During training, each lever is associated with either 0.75 mg/kg cocaine in 10% w/v ethanol or saline injections (i.p. 15 minutes pre-session). The subjects learn to discriminate which injection is given in order to determine which lever is correct for each session. Training is continued until the subjects begin each session on the correct lever for four consecutive sessions.

Testing: Generalization tests are conducted twice a week. Between test sessions, animals are provided continued training with ethanol/cocaine and saline injections. On test sessions, responding on both levers is reinforced. Tests with 0.75 mg/kg cocaine in 10% w/v ethanol and saline are conducted at the beginning of each dose-effect curve determination. A complete dose-effect curve is obtained for each test drug. Test injections are given i.p. 15 minutes before start of the test session. Tests are conducted with the following three drugs coadministered with 10% w/v ethanol: dexmethylphenidate HCl (d-MPH), d,l-methylphenidate HCl (d,l-MPH), and cocaine. Test solutions are prepared by dissolving the test material in 10% w/v ethanol to yield an injection volume of 1 ml/kg. All injections are given i.p. 15 minutes before training and testing sessions.

Results: The degree of ethanol/cocaine-like discriminative stimulus effects is reflected in the percentage of responses during test sessions on the lever associated with ethanol/cocaine during training sessions. These values are calculated for each rat and averaged for the group. As with ethanol/cocaine, ethanol/d,l-MPH produces 100% ethanol/cocaine-lever response rates at one or more doses that do not decrease rates of response compared with those on saline control test sessions. Ethanol/d,l-MPH is equipotent when compared to ethanol/cocaine for both discriminative stimulus and response rate effects. Ethanol/d-MPH is half as potent as compared to ethanol/d,l-MPH.

What is claimed:

1. A method of reducing co-abuse of a methylphenidate drug and ethyl alcohol comprising:
identifying a patient or patient group abusing the methylphenidate drug and ethyl alcohol; and
providing to the patient or patient group a methylphenidate drug substantially free of l-threo methylphenidate.

2. The method of claim 1 wherein said methylphenidate drug is made available orally, intravenously, parenterally, via an aerosol or gaseous suspension, or transdermally.

3. The method of claim 2 comprising up to 10 mg of methylphenidate drug.

4. The method of claim 2 comprising up to 5 mg of methylphenidate drug.

5. The method of claim 2 comprising up to 3 mg of methylphenidate drug.

6. The method of claim 2 comprising from about 0.1 mg to about 100 mg of methylphenidate drug.

7. The method of claim 1 wherein said patient has or is suspected of having ADD or ADHD.
